(19) United States
(12) Patent Application Publication
(10) Pub. No.: US 2009/0186052 A1
(43) Pub. Date: Jul. 23, 2009

(54) USE OF BOTULINUM TOXIN FOR PORLONGED LOCAL DESENSITIZATION

(75) Inventor: Roland Cherif-Cheikh, Castelldefels (Barcelona) (ES)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Societe de Conseils de Recherches et D'Applications Scientifiques (S.C.R.A.S.), Paris (FR)

(21) Appl. No.: 11/722,459

(22) PCT Filed: Dec. 21, 2005
(86) PCT No.: PCT/FR2005/003209

§ 371 Date: Jun. 21, 2007

(30) Foreign Application Priority Data
Dec. 21, 2004 (FR) 0413650

(51) Int. Cl. A61K 39/08

(52) U.S. Cl. 424/239.1

(57) ABSTRACT

The invention concerns the use or a fragment of botulinum toxin having said toxin activity for preparing a medicine for reversibly desensitizing an area of the skin, said skin area being designed to receive injections. Said injections can be related, for example, to repeated delivery of insulin, growth hormones or other medicines in injectable form.

This invention relates to the use of botulinum toxin for prolonged local desensitization.

Botulinum toxin, in particular type A botulinum toxin (Dysport® marketed by Ipsen or Botox® marketed by Allergan), has been used since the 1980s in humans for the treatment of many and varied diseases/disorders. Among the diseases/disorders which can be treated with botulinum toxin, there may be mentioned, amongst others, muscular disorders (for example blepharospasm, adult or child spasticity or also torticollis), migraine, pain of muscular origin, neuropathic pain, diabetes, hyperhidrosis (or excessive perspiration), hypersalivation or even wrinkles.

The applications of botulinum toxins known to date have related to their standard intra-muscular administration as described in the treatments mentioned. Pain reduction for the applications concerned, as in all the therapeutic applications of these toxins, is obtained by temporary muscle paralysis after injection into the muscles, i.e. by blocking muscular contractions over a certain period of time. Generally therefore, pain is controlled indirectly by means of controlling muscular spasms or contractions.

There are a number of pathologies where localised pain affects the skin and/or subcutaneous tissue which could advantageously benefit from a prolonged and repeatable treatment. This is the case for example of pain caused by injection of controlled release forms, such as for example autogel injections, subcutaneous inserts and implantations (pumps, pacemakers, etc.). Similarly, certain therapeutic treatments require the regular injection of medicinal substances, for example in the form of daily injections. This is the case in particular of treatments for insulin-dependant diabetes, where the patient self-injects a minimum of once a day, but more usually after each meal, with a dose of insulin. This is the case for example in certain treatments which require daily injections of growth hormone. It is also the case for example in certain treatments for cancer which require repeated injections of an anti-cancer agent. It is equally the case for repeated blood collections such as those associated with blood sugar analyses. This is often very painful for patients, some of whom eventually develop an aversion for punctures and all forms of injections.

All above-mentioned pain is termed, according to the present invention, repeated, repetitive or specific induced pain. This is pain arising from an external intervention, by a stimulus or by an invasive trauma affecting the dermis or skin or subcutaneous tissue. The pain can for example be due to the injection itself, in particular incision of the dermis by the needle, or it can also be due to the injected product itself.

In these cases, as for any other sustained or specific repetitive induced pain affecting the skin and subcutaneous tissue, it would appear desirable to have available a sustained and localised anaesthetic treatment. However, to the Applicant's knowledge, no effective and durable solution has to date been found to remedy these problems.

In fact the Applicant has just discovered that botulinum toxin can be used for preparing a medicament intended to desensitize an area of skin in a reversible fashion, said area of skin being subject to repeated, repetitive or specific induced pain caused by repeated punctures or injections. Thus, said repeated, repetitive or specific induced pain is avoided or treated. This pain can in particular correspond to a treatment, with repeated punctures (linked for example to repeated administrations of insulin, growth hormone, chemotherapy agents or other medicaments in injectable form or also to punctures for blood collections, (for example for measuring blood glucose levels) or for putting implants or implantable devices in place (for example pumps, pacemakers, etc), or systems for the administration of therapeutic substances (for example perfusions).

The subject of this invention is therefore the use of botulinum toxin or a fragment of botulinum toxin possessing the activity of said toxin for preparing a medicament intended to desensitize in a reversible fashion an area of skin, said area of skin being subject to repeated, repetitive or specific induced pain caused by repeated punctures or injections.

According to this novel use according to the invention, the botulinum toxin is preferably injected directly by intradermal, transdermal or subcutaneous route into the treatment area and not by the intramuscular route as normal. If appropriate, this administration can take the form of applying a patch or a composition for topical application (for example, cream, gel, lotion, etc.) comprising botulinum toxin.

The botulinum toxin used for the preparation of a medicament according to the invention is preferably chosen from the botulinum toxins of type A (including A₁, A₂ and A₃), B, C (including C₁ and C₂), D, E, F or G. Preferably, it is chosen from botulinum toxin of type A, B or F. Even more preferentially, it is chosen from botulinum toxin of type A or B; in particular, it is botulinum toxin of type A. Alternatively, it can be a fragment of botulinum toxin possessing the activity of said toxin, or also of a analogue of botulinum toxin possessing the activity of said toxin.

Moreover, when a botulinum toxin of type A, B, C, D, E, F or G is used for the preparation of a medicament according to the invention, said toxin can be presented in the form of a complex comprising botulinum toxin or in the free form (i.e. free of any complexing protein).

According to the invention, the medicament prepared can be a lyophilized powder comprising botulinum toxin (in which case the doctor will reconstitute the solution with water or an aqueous saline solution before injecting the patient) or also an injectable solution comprising said toxin (such as for example as that described in Patent Application WO 00/15245).

According to a preferential variant, this invention relates to the use of botulinum toxins or a fragment of botulinum toxin possessing the activity of said toxin for preparing a medicament intended to desensitize in a reversible fashion an area of skin, said area of skin being subject to repeated, repetitive or specific induced pain caused by administrations of insulin, growth hormone, peptides in the form of “autogel”, chemotherapy agents or other medicaments in injectable form or also caused by punctures for blood collections for measuring blood glucose level.

By peptides in “autogel” form, is meant in the context of this invention a delayed-release formulation of peptides, such as for example the formulation of somatostatin analogue (Somatuline in “autogel” form, 60, 90 or 120 mg).

By insulin, is meant any insulin, whether with a very rapid, rapid, intermediate, slow or very slow action, or also a mixture of such insulins. By very rapid-acting insulin is meant in particular lispro insulin (i.e. the insulin analogue constituting the active ingredient of Humalog®). By rapid-acting insulin, is meant in particular recombinant human insulins such as the active ingredient of Humulin® R (Eli Lilly) or Novolin® ge Toronto (Novo Nordisk). By intermediate insulin, is meant in particular Neutral Protamine Hagedorn insulin or NPH insulin (and for example the active ingredient of Humulin® N or Humulin® L (Eli Lilly) or Novolin® ge NPH or Novolin® ge Lente (Novo Nordisk)). By slow- or very slow-acting insulin is meant in particular glargine insulin (i.e. the insulin analogue constituting the active ingredient of Lantus®), the insulins associated with Zn²⁺ ions or the active ingredient of Humulin® U (Eli Lilly) or Novolin® ge Ultralente (Novo Nordisk).

By growth hormone, is meant any natural or synthetic growth hormone (and for example a recombinant growth hormone), and in particular the growth hormone marketed in Europe under the trade mark NutropinAq®.

By repeated punctures or injections is meant daily or weekly or bimonthly punctures or injections, preferably taking place in a perimeter of skin of the patient comprised between 1 and 5 cm². Preferably the repeated punctures or injections are subcutaneous punctures or subcutaneous injections.

By the expression “desensitize an area of skin in a reversible fashion”, is meant in the context of the present invention, local anaesthesia of the area of skin. Thus, this invention does not aim to treat the pain but to anaesthetise an area of skin.

According to the invention, the desensitized area of skin can be produced by sub-cutaneous injection of botulinum toxin at a single point, or preferably, by the sub-cutaneous injection of botulinum toxin at several points defining a geometrical figure (for example an equilateral triangle, a square, a regular pentagon, a regular hexagon, etc.) or evenly distributed over an area of skin (for example a network of distributed points).

Preferably, the treated area is as small as possible and limited to the needs of pain control. In the case of repeated injections for example, this can be an area from 1 to 5 square centimetres of associated skin surface, preferably from 1 to 3 cm² or not to an area from 1 to several centimetres in depth of subcutaneous tissue.

In the particular case of persons who are to undergo punctures for blood collections for measuring blood glucose levels, it would be possible for example to envisage carrying out one or more injections of botulinum toxin in one or more fingers in order to desensitize the areas intended to receive the corresponding needle or lancet punctures.

Preferably, the local desensitization produced by the injection of botulinum toxin will last for at least one month, and more preferentially at least 2, 3 or even 6 months.

Variation of the dose and the dilution will allow adjustment of the activity and the diffusion of the botulinum toxin and consequently of the part of the skin and subcutaneous tissues desensitized, as well as the intensity of the treatment.

Preferably the injected doses are comprised between 10 and 200 Units of botulinum toxin, more preferentially 20 to 150 Units of botulinum toxin, even more preferentially 50 to 100 Units of botulinum toxin.

In order to avoid lesions to the tissues close to the area treated by the botulinum toxin and undergoing the regular punctures, there will preferably be a regular change of treatment area (in any case at the latest at the time when the desensitization produced by the botulinum toxin ceases to produce its effects); for example, in the case of the botulinum toxin of type A, it can be expected to change area approximately 3 months after administration of the toxin. In this situation therefore, the appropriate dose of botulinum toxin will be administered to the new area due to be desensitized 1 to 7 days before receiving the regular punctures, in order to leave the botulinum toxin the time to produce its effects.

The treatment will be repeated according to the needs of the patient. It is possible, however, that this help with local tolerance at the start of the treatment may have been sufficient and that the patient does not need this treatment any more, or that it may be possible to reduce the dose progressively. Preferably therefore these local injections of botulinum toxin are carried out a short time before (for example from 1 to 10 days) or at the beginning (i.e. the on the first day) of routine treatments by injection or also a short time before (for example from 1 to 10 days) or at the time (i.e. the same day) of carrying out a painful subcutaneous implantation procedure of a device such as a pump, pacemaker, mammary prosthesis or other.

In the case of other pathological pain at this level, the treatment can also be a curative response and not merely a preventative treatment.

This use according to the invention excludes treatment of pain caused by formalin or arthritis or a tumour of any kind or a burn or migraine. The use according to the invention also excludes the treatment of inflammatory pain or post-operative or visceral pain.

According to a variant of the invention, the patient of which an area of skin is desensitized in a reversible fashion can be a animal. Preferably, said patient will be a human being (male or female).

Unless defined otherwise, all the technical and scientific terms used here have the same meanings as those normally understood by an ordinary specialist in the field to which this invention belongs.

The following examples are given in order to illustrate the above procedures and must not under any circumstances be considered as limiting the scope of the invention.

### EXAMPLES

### Example 1

A diabetic patient must regularly inject himself with insulin by the subcutaneous route. The patient receives 40 Ipsen units of botulinum toxin subcutaneously (Dysport®; supplier: Ipsen Limited, United Kingdom; preparation according to the manufacturer's recommendations) distributed evenly at 4 points defining a 2 cm square marked on the skin of the patient. After a few days, the subcutaneous injections of insulin carried out in the square pretreated with the botulinum toxin become much less painful for the patient. Three months later, the procedure is repeated, choosing another square of skin as the area to be desensitized.

### Example 2

A child of 8 years of age is prescribed daily subcutaneous injections of growth hormone. One week before starting the treatment with the growth hormone, 30 Ipsen units of botulinum toxin (Dysport®, supplier: Ipsen Limited, United Kingdom; preparation according to the manufacturer's recommendations) are distributed evenly and injected by subcutaneous route at 4 points defining a 2 cm square marked on the skin of the patient. A week later, the daily subcutaneous injections of growth hormone are carried out within the limits of the square pretreated with the botulinum toxin without the child feeling real pain associated with the injections. Three months later, the procedure is repeated, choosing another square of skin as the area to be desensitized.

### Example 3

A diabetic patient is treated with 10 Ipsen units of botulinum toxin Dysport® or using the same micro volume, with physiological serum placebo in different injection sites in order to take a drop of blood with a lancet type device making it possible to then measure the blood glucose level, for example at the fingertips.

The injection sites of Dysport® and placebo are then exposed and assessed by the patient according to a pain scale of 1 to 6.

The result of this study was a significant difference with a reduction of the pain experienced at the sites treated by Dysport® (botulinum toxin) over a period of 3 months and more.

### Example 4

A patient suffering from acromegaly is treated with a delayed-action form of Autogel by sub-cutaneous route at a rate of one injection of 120 mg of lanreotide every month (Somatuline 120 mg acquired from Ipsen). He assess the pain of the injection according to a scale. The usual injection sites are treated with an injection of 40 Ipsen units of Dysport® botulinum toxin and the pain related to the treatment is evaluated by difference to the reference.

It appears from this study that the 6 consecutive injections in the treated area, i.e. over 6 months of treatment, are significantly perceived as less painful than before the administration of Dysport®.

1. A method for reversibly desensitizing an area of skin subjected to repeated, repetitive or specific induced pain caused by repeated punctures or injections, comprising applying a medicament comprising botulinum toxin or a fragment of botulinum toxin possessing the activity of said toxin.

2. The method of claim 1, wherein the pain is caused by the administration of insulin, growth hormone, peptides in the form of autogel chemotherapy agents or other medicaments in injectable form or also caused by punctures for blood collections for measuring blood glucose level.

3. The method of claim 1 or 2, wherein the botulinum toxin is a botulinum toxins of type A, B, C, D, E, F or G.

4. The method of claim 3, wherein the botulinum toxin used is a botulinum toxins of type A, B or F.

5. The method of claim 4, wherein the botulinum toxin used is a botulinum toxin of type A or B.

6. The method of claim 5, wherein the botulinum toxin used is a botulinum toxin of type A.

7. The method of claim 1, wherein the medicament is administered by intradermal, transdermal or subcutaneous route.

8. The method of claim 1, wherein the desensitization caused lasts at least one month.

9. The method of claim 8, wherein the desensitization caused lasts at least two months.

10. The method of claim 9, wherein the desensitization caused lasts at least three months.
